(19) United States
(12) Patent Application Publication
(10) Pub. No.: US 2004/0158322 A1
(43) Pub. Date: Aug. 12, 2004

(54) Intraocular lens system

(76) Inventor: Jin Hui Shen, Nashville, TN (US)

Correspondence Address: MERCHANT & GOULD PC, P.O. BOX 2903, MINNEAPOLIS, MN, 55402-0903, US

(21) Appl. No.: 10/474,988

(22) PCT Filed: Apr. 17, 2002
(86) PCT No.: PCT/US02/11956

(51) Int. Cl. A61F 2/16

(52) U.S. Cl. 623/6.33; 623/6.34

(57) ABSTRACT

A system and intraocular lens system for implantation in an eye having a lens capsule and lens substance contained in the lens capsule. In one embodiment of the present invention, the intraocular lens system has a frame having a center, a first optical element with a focal power and a second optical element with a focal power. The first optical element has a center, a surface and an edge, wherein the first optical element is coupled to the frame at its edge such that the center of the first optical element is at a distance from the center of the frame. The second optical element has a center, a surface and an edge, wherein the second optical element is coupled to the frame at its edge such that the center of the second optical element is at a distance from the center of the frame. The first optical element and the second optical element at a predetermined state are positioned such that the distance between the first optical element and the center of the frame and the distance between the second optical element and the center of the frame are substantially same, and the surfaces of the first optical element and the second optical element partially overlap to form a combined lens with an effective focal power that is different from either of the focal power of the first optical element and the focal power of the second optical element.

### CROSS-REFERENCE TO RELATED APPLICATION

This application claims priority to U.S. Provisional Application Serial No. 60/284,359, which was filed on Apr. 17, 2001, in the United States Patent and Trademark Office.

### BACKGROUND OF THE INVENTION

1. Field of the Invention

The present invention generally relates to an intraocular lens system. More particularly, the present invention relates to an intraocular lens system that has an adjustable focal length responsive to changes of the diameter of the lens capsule

2. Description of the Related Art

Accommodation, or a change in the focus of the human lens, is a consequence of the ability of the lens to change its shape by contracting the capsule. This contraction function is what normally changes the shape of lens capsule in response to the need to accommodate.

The crystalline lens is one of the main optical elements in human vision. It provides the focus adjustment function in the eye. As shown in FIGS. 1(A) and (B) from Von Helmholtz, Hb. D. Physiol. Optik., Leipzig, 1(1856); 2nd ed., Hamburg, 175 (1896), the lens 100 has a capsule 102 and lens substance 104. The lens 100 is suspended by zonules 106 from the ciliary processes 108. Normally, when the lens 100 is at a non-accommodating condition as shown in FIG. 1(A), which means the eye is focused at a distance, the ciliary muscle 108 is at a relaxed condition. The shape of the lens 100 is relatively flat, which is determined by its own natural elasticity, and the lens 100 now has a lower focal power. When the eye looks at objects a short distance away as shown in FIG. 1(B), however, the ciliary muscle 108 contracts, and the lens 100 tends to accommodate. For this to happen, the lens 100 has to increase its thickness. Correspondingly, there are a decrease in the diameter of the lens 100 and a decrease in the anterior and posterior surface radii, which are determined by the natural shape of capsule 102. As shown in FIG. 1(B), in the act of accommodation, the anterior surface of the lens 100 becomes more convex axially, and the posterior surface of the lens 100 also becomes more convex. Consequently, a higher focal power for the lens 100 is created. The parameter changes during lens accommodation are listed in table I of Sir Stewart Duke-Elder, and David Abrams, System of Ophthalmology, Vol V, Ophthalmic Optics and Rferaction, St. Louis, The C. V. Mosby Company, 1970, P177.

TABLE 1
Lens parameter changes with accommodation

| | Unaccommodated | Accommodated | |
|---|---|---|---|
| | condition | condition | Reference |
| Refracting power | +19.11D | +33.06D | ¹ |
| Focal length | 43.707 mm | 33.785 mm | ² |
| | 69.908 mm | 40.416 mm | 2 |
| Radius of lens surface | 11.62 mm | 6.90 mm | 3 |
| | 12 mm | 5.0 mm | 3 |
| Thickness of the lens | 3.66 mm | 4.24 mm | ³ |
| | 3.84 mm | 4.20 mm | 3 |
| Lens equatorial diameter | | | |
| 15 yr. | 9 mm | 8 mm | ⁴ |
| (lens from different age) | | | |
| 43 yr. | 10.4 mm | 9.4 mm | 4 |
| 63 yr. | 10.8 mm | 9.8 mm | 4 |

As people age, the amplitude of accommodation is gradually reduced due to changes in the lenticular factors such as a decrease in the elasticity modulus of the capsule, an increase in the elasticity modulus of the lens substance, a flattening of the lens, or a combination of them. FIG. 2, by Fincham, Brit. J. Ophthal, 35,381 (1951), J. Physiol., 128, 99 (1955), and Vision Res., 1,425 (1962), shows presbyopic changes in the amplitude of accommodation due to changes with age in the lens.

When a person ages, the substance of the person's natural lens gradually hardens, and may lose its accommodation function. Additionally, the person's vision is also reduced by cataract formation. Cataract surgery is then necessary to restore vision.

In modern cataract surgery, the cataractous substance of the lens is removed through an opening in the lens capsule. The now empty capsule of the lens is retained. The surgeon then replaces the lens contents with an artificial lens, which is positioned in the empty capsule. A typical procedure for a cataract surgery includes providing a opening at limbus, removal of the front portion of the lens capsule, ultrasonic fragmentation of the hard lens substance (nucleus), and implantation of an artificial intraocular lens.

Intraocular lenses (hereinafter referred as “IOL”) are high optical quality lenses made of synthetic material such as PMMA, silicone, hydrogel or the like. The diameter of an IOL is normally 5 to 7 mm, and the lens dioptric power is matched to the need of the patient. Each IOL has two spring-like haptics, or loops, attached to the optic. When the IOL is inserted inside the lens capsule, the haptics help to position the optic lens in the center. Haptics material are PMMA, polypropylene, or polyamide. There are varieties of haptic designs among different IOLs. Some of the configurations are show in FIG. 3. For examples, IOL 301 has optic 302 and haptics 304, where haptics 304 are J-shaped loops. Moreover, IOL 311 has haptics that are C-shaped loops, IOL 321 has haptics that are lone J-shaped loops, and IOL 331 has haptics that are closed loops.

Visual function following cataract and IOL implant surgery generally is good. However, among other things, a major disadvantage is the loss of accommodative capability that a natural lens can offer because the artificial intraocular lens has a fixed focusing power.

Previous research by R. F. Fisher, The significance of the shape of the lens and capsular energy changes in accommodation, J. Physiol. (1969), 201, pp. 21-47, has showed that after extraction of the cataractous lens contents, the lens capsule still retains a certain level of the accommodative capability.

Efforts have been made to restore accommodation after cataract and implant surgery, which can be divided into the following categories:

1) Refill the lens with a synthetic material. This technique was first introduced by Kessler in Experiments in refilling the len, Arch Ophthalmol 1964; 71:412-7. Efforts have been continued to improve the technology around the world, for examples, by a research group at Bascom Palmer Eye Institute, University of Miami, Fla., as shown in E. Haefliger, J-M. Parel, F. Fantes, E. W. D. Norton, D. R. Anderson, R. K. Forster, E. Hernandez, W. J. Feuer, Accommodation of an endocapsular silicone lens (phacoersatz) in the nonhuman primate, Ophthalmology 94:471477, 1987, and a research group in Japan as shown in O. Nishi, K. Nishi, C. Mano, M. Ichihara, T. Honda, Controlling the capsular shape in lens refilling, Arch Ophthalmol, 1997; 115:507-510; Y. Sakka, T. Hara, Y. Yamada, T. Hara, F. Hayashi, Accommodation in primate eyes after implantation of refilled endocapsular ballon, American Journal of Ophthalmology 121:210-212, 1996. The normal procedure for this technique includes the steps of removing the crystalline lens through a small anterior capsular hole, and refilling the capsular bag with either pre-cured silicone gel, or an inflatable endocapsular balloon. All of these studies showed that the refilled lens recovered accommodation to some extent, but the amount was not sufficient to be clinically useful.

2) Bifocal or multifocal intraocular lens. Bifocal or multifocal IOLs were first introduced clinically in 1987 by Keates et al. as shown in Keates R. H., Pearce J. L., Schneider R. T: Clinical results of the multifocal lens, J. Cataract Refract Surg. 13:557-560, 1987. Currently, several different types of multifocal IOL have been developed, including the multizone bifocal lens, as shown in Percival P., Indications for the multizone bifocal implant, J. Cataract Refract Surg. 16:193-197, 1990; Jacobi P. C., Konen W., Effect of age and astigmatism on the AMO Array multifocal intraocular lens, J. Cataract Refract Surg. 21:556-561,1995, the aspherical multifocal IOL, as shown in Christie B. Nordan L. Chipman R. Gupta A., Optical performance of an aspheric multifocal intraocular lens. J. Cataract Refract Surg. 17:583-591,1991, and the diffractive multifocal IOL, as shown in Bellucci R. Giardini P., Pseudoaccommodation with the 3M diffractive multifocal intraocular lens: A refraction study of 52 subjects. J. Cataract Refract Surg. 19:32-35,1993, Holladay J. T., van Dijk H., Lang A., et al., Optical performance of multifocal intraocular lenses, J. Cataract Refract Surg. 1990, 16:413-422, Olsen T., Corydon L., Contrast sensitivity in patients with a new type of multifocal intraocular lens, J. Cataract Refract Surg. 16:42-46, 1990, Auffarth G. U., Hunold W., Wesendahl T. A., Mehdom E., Depth offocus andfunctional results in patients with multifocal intraocular lenses: A long-term follow-up, J. Cataract Refract Surg. 19:685-689,1993. Nevetheless, these IOLs can only give a patient two focus points and/or a limited focus range, and at each focus point, the patient can only get half of the incoming light energy. Consequently, at each focus distance, the images the patient has are blurry.

3) Accommodative intraocular lens. Several groups have been working along this line of research. For examples, one is in Japan, as shown in Hara T, Hara T., Yasuda A., Yamada Y., Accommodative intraocular lens with spring action. Part 1. Design and placement in an excised animal eye, Ophthalmic Surg. 1990; 21:128-133, and the other in the Netherlands, as shown in Cumming J. S., Kammann J., Experience with an accommodating IOL, J. Cataract Refract Surg. 1996; 22:1001. In both studies, a movable optical lens is utilized in the direction of the axis of the eye, which is controlled by the ciliary muscle. While there was a limited amount of accommodative function shown, again no full accommodation was restored.

Recently, an accommodative IOL was proposed by Oliver Findl, M. D., of Vienna, Austria, as shown in Cumming J. S., Kammann J., Experience with an accommodating IOL, J. Cataract Refract Surg. 1996; 22:1001. As shown in FIG. 4, in Dr. Findl's IOL design, a fixed focus lens 402 is held by two pieces 404, 406 of ridged plastic holder, and the connection 408 between each plastic holder 404 or 406 and the lens 402 is flexible. When the ciliary muscle contracted, the IOL 400 will move forward. By this design, up to 2.5D of the accommodation has been achieved. Still, no full scale of accommodation is available.

Therefore, there is a need to develop a new intraocular lens system that is responsive to changes of the diameter of the lens capsule and can provide better accommodation capability than the currently available techniques.

### SUMMARY OF THE INVENTION

In one aspect, the present invention relates to an intraocular lens system for implantation in an eye having a lens capsule and lens substance contained in the lens capsule. In one embodiment of the present invention, the intraocular lens system has a frame having a center, a first optical element with a focal power and a second optical element with a focal power. The first optical element has a center, a surface and an edge, wherein the first optical element is coupled to the frame at its edge such that the center of the first optical element is at a distance from the center of the frame. The second optical element has a center, a surface and an edge, wherein the second optical element is coupled to the frame at its edge such that the center of the second optical element is at a distance from the center of the frame. The first optical element and the second optical element at a predetermined state are positioned such that the distance between the first optical element and the center of the frame and the distance between the second optical element and the center of the frame are substantially same, and the surfaces of the first optical element and the second optical element partially overlap to form a combined lens with an effective focal power that is different from either of the focal power of the first optical element and the focal power of the second optical element.

In one embodiment of the present invention, the frame is elastic and adapted to be in contact with the lens capsule of the eye. When the lens capsule of the eye presses the frame in a direction toward to the center of frame, the motion of the frame causes the first optical element and the second optical element to move toward the center of the frame from the first predetermined state to a second predetermined state in which the first optical element and the second optical element are substantially overlapping to each other so as to be substantially concentric with the center of the frame to form a combined lens with an effective focal power that is different from the effective focal power of the combined lens at the first predetermined state. In general, the effective focal power of the combined lens at the second predetermined state is larger than the effective focal power of the combined lens at the first predetermined state.

The frame can have various configurations. For examples, the frame can comprise a closed-loop structure. The closed-loop structure can be symmetrical to the center of the frame. In one embodiment, the frame is an annular structure. In another, the frame is a multi-round-cornered structure. Alternatively, the frame can comprise an open-loop structure.

The intraocular lens system further includes an optional supporting portion that is attached to the frame. The supporting portion has a first end, a second end, and a surface defined between the first end and the second end, wherein the surface of the supporting portion has a curvature corresponding to the curvature of the lens capsule so as to allow the supporting portion to be positioned between the lens capsule and the frame.

The first optical component comprises a lens that has a thickness. The surface of the lens varies as a mathematical function of the distance from the center of the lens. The mathematical function can be a Gaussian distribution, a distribution that can be represented by a spherical-harmonic approximation or the like. Likewise, the second optical component comprises a lens that has a thickness. The surface of the lens varies as a mathematical function of the distance from the center of the lens. The mathematical function can be a Gaussian distribution, a distribution that can be represented by a spherical-harmonic approximation or the like. Moreover, the lens utilized to practice the present invention may further have a ridged edge portion, where the dimension of the ridged portion is comparable to the thickness of the lens so that when the first optical component and the second optical component overlap to each other, the ridged portions of the lenses are in contact to avoid the optical sensitive portions of the lenses to rub against each other. While the lenses utilized to practice the present invention in one intraocular lens system can be different, they are chosen to be substantially identical to each other for the embodiments disclosed in this specification.

In another aspect, the present invention relates to an intraocular lens system for implantation in an eye having a lens capsule and lens substance contained in the lens capsule. In one embodiment of the present invention, the intraocular lens system includes a frame having a center and a plurality of lenses. Each of the plurality of lenses has a center, a surface and an edge, wherein the surface of each lens varies as a Gaussian function of the distance from the center of the lens. Each of the plurality of lenses is coupled to the frame at the edge such that the center of each lens is at a distance from the center of the frame, wherein the plurality of lenses at a first predetermined state are positioned such that the distances between the center of each lens and the center of the frame are substantially same, and the surfaces of the lenses partially overlap to form a combined lens with an effective focal power.

In one embodiment of the present invention, the frame is elastic and adapted to be in contact with the lens capsule of the eye. When the lens capsule of the eye presses the frame in a direction toward to the center of frame, the motion of the frame causes the plurality of the lenses to move toward the center of the frame from the first predetermined state to a second predetermined state in which the plurality of lenses are substantially overlapping to each other so as to be substantially concentric with the center of the frame to form a combined lens with an effective focal power that is different from the effective focal power of the combined lens at the first predetermined state. In general, the effective focal power of the combined lens at the second predetermined state is larger than the effective focal power of the combined lens at the first predetermined state.

The plurality of lenses is positioned such that the centers of the plurality of lenses are symmetrical about the center of the frame. The total number of the plurality of lenses is N, wherein N is an integer that is not smaller than 2.

In yet another aspect, the present invention relates to an intraocular lens system for implantation in an eye having a lens capsule and lens substance contained in the lens capsule. In one embodiment of the present invention, the intraocular lens system includes a frame having a center and a plurality of lenses, total number being N. Each of the plurality of lenses has a center, a surface and an edge, wherein the surface of each lens varies as a mathematical function of the distance from the center of the lens. Each of the plurality of lenses is coupled to the frame at the edge such that the center of each lens is at a distance from the center of the frame, wherein the plurality of lenses at a first predetermined state are positioned such that the distances between the center of each lens and the center of the frame are substantially same and the surfaces of the lenses partially overlap to form a combined lens with an effective focal power.

In one embodiment of the present invention, the frame is elastic and adapted to be in contact with the lens capsule of the eye. When the lens capsule of the eye presses the frame in a direction toward to the center of frame, the motion of the frame causes the plurality of the lenses to move toward the center of the frame from the first predetermined state to a second predetermined state in which the plurality of lenses are substantially overlapping to each other so as to be substantially concentric with the center of the frame to form a combined lens with an effective focal power that is different from the effective focal power of the combined lens at the first predetermined state. In general, the effective focal power of the combined lens at the second predetermined state is larger than the effective focal power of the combined lens at the first predetermined state.

The plurality of lenses is positioned such that the centers of the plurality of lenses are symmetrical about the center of the frame. The total number of the plurality of lenses is N, wherein N is an integer that is not smaller than 2.

Moreover, for each of the plurality of lenses, the surface of each lens varies as a mathematical function of the distance from the center of the lens, wherein the mathematical function can be a Gaussian distribution, a distribution that can be represented by a spherical-harmonic approximation, a distribution that is symmetrically decreasing with the distance from the center of lens or the like.

In a further aspect, the present invention relates to an intraocular lens system for implantation in an eye having a lens capsule and lens substance contained in the lens capsule. In one embodiment of the present invention, the intraocular lens system has a plurality of lenses to form a combined lens, where each lens has a Gaussian surface curvature that can be described as z=t×exp[−α(x²+y²)] and in an eccentric distance from the center of the combined lens. The focal length of the combined lens can change with the change of the eccentric distance in response to the change in the diameter of the lens capsule, which in turn may generate the accommodation effect. Alternatively, an intraocular lens system according to the present invention can have a plurality of lenses each having a surface curvature other than a Gaussian surface curvature such as sine, cosine, exponential or the like.

These and other aspects will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 is a perspective view of (A) an unaccommodated lens; and (B) an accommodated lens.

FIG. 2 is a chart showing presbyopic changes in the amplitude of accommodation due to changes with age in the lens.

FIG. 3 illustrates several configurations of the IOL in the prior art.

FIG. 4 illustrates an accommodative intraocular lens studied by Oliver Findl. Up to 2.5 diopter of accommodative power has been reported.

FIG. 5 illustrates an IOL that has six eccentric lenses partially overlapping to form a combined lens in one embodiment of the present invention.

FIG. 6 shows that a combined lens having concentric overlapped lenses at pressed state have more focus power than a combined lens having eccentric overlapped lenses at a relaxed state in one embodiment of the invention.

FIG. 6A schematically shows an IOL according to one embodiment of the present invention.

FIG. 7 illustrates the surface of an optical lens that can be utilized to practice the present invention.

FIG. 8 illustrates an IOL having symmetrically overlapped six lenses with the off-center distance of D in one embodiment of the invention.

FIG. 9 shows a profile of a single Gaussian lens with t=2.1×10⁵ m, and a=7.5×10⁵ m⁻².

FIG. 10 illustrates an IOL having a different configuration according to another embodiment of the present invention.

FIG. 11 illustrates an experimental set up for measuring the force to move the Gaussian lenses the required distance employed in one embodiment of the invention.

FIG. 12 shows cross-sectionally an IOL positioned in the lens capsule, in one embodiment of the present invention.

FIG. 13 illustrates an optical lens that prevents the central portion of the lens from rub each other: (A) a perspective view of the ridged ring edge; (B) a perspective view of the optical lens with a frame; (C) a cross-sectional view of the optical lens with the ridged edge portion; and (D) a top view of (C) according to one embodiment of the present invention.

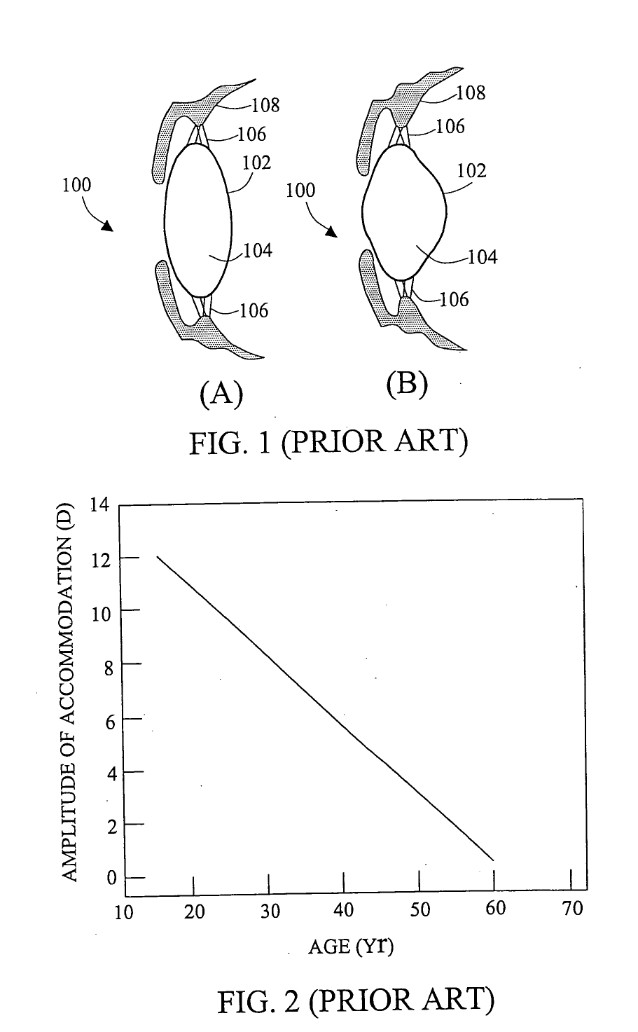

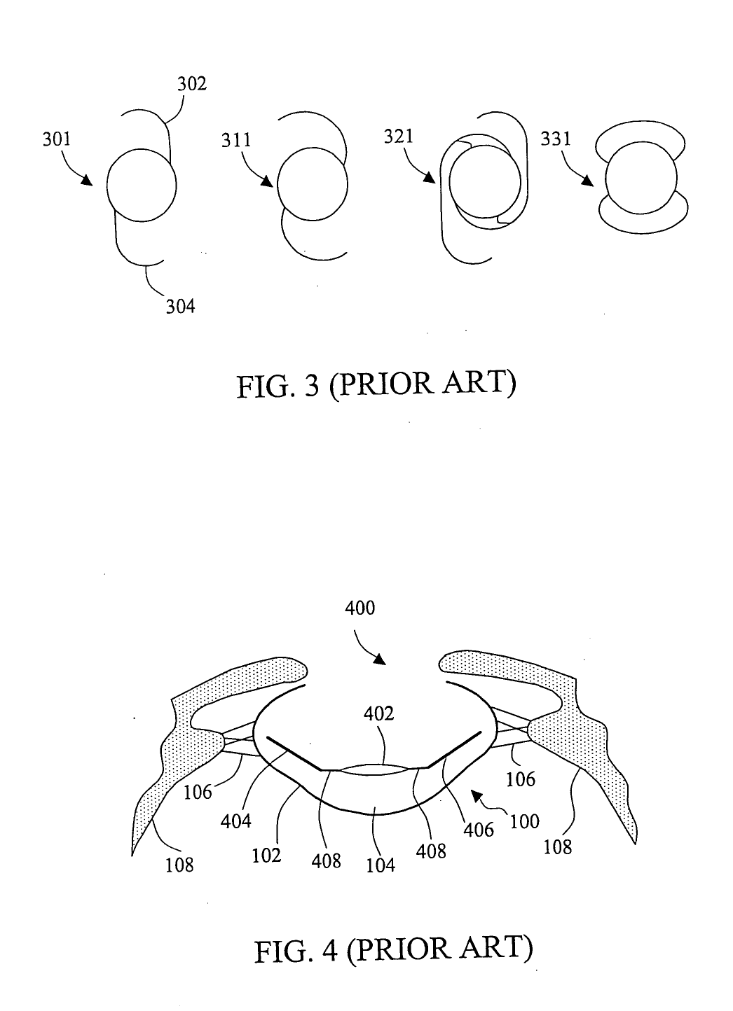

### DETAILED DESCRIPTION OF THE INVENTION

Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

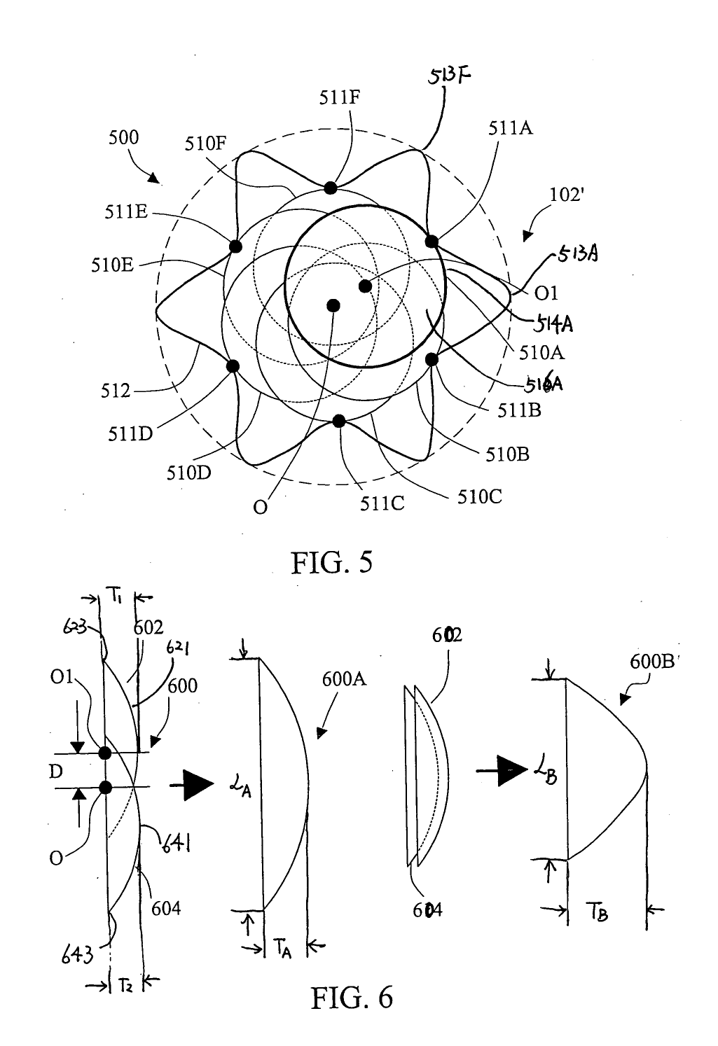

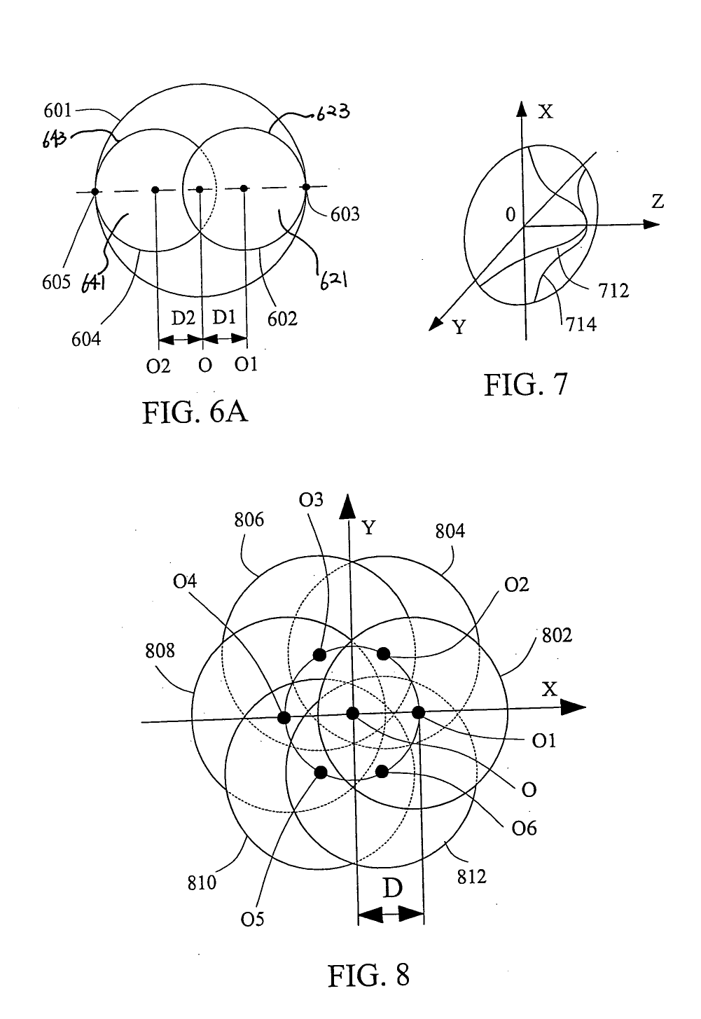

In one aspect, referring in general to FIGS. 5-13, the present invention relates to an intraocular lens system for implantation in an eye having a lens capsule and lens substance contained in the lens capsule. In one embodiment of the present invention as particularly shown in FIGS. 6 and 6A, an intraocular lens system 600 has a frame 601 having a center O, a first optical element 602 with a focal power and a second optical element 604 with a focal power.

The first optical element 602 has a center O₁, a surface 621 and an edge 623. The first optical element 602 is coupled to the frame 601 at its edge 623 at a position 603 such that the center of the first optical element 602, O₁, is at a distance, D₁, from the center of the frame 601, 0. In other words, the first optical element 602 is positioned eccentrically from the center of the frame 601, O at a distance D₁.

The second optical element 604 has a center O₂, a surface 641 and an edge 643. The second optical element 604 is coupled to the frame 601 at its edge at a position 605 such that the center of the second optical element 604, O₂, is at a distance, D₂, from the center of the frame 601, O. In other words, the second optical element 604 is positioned eccentrically from the center of the frame 601, O at a distance D₂.

As shown in FIG. 6A, the first optical element 602 and the second optical element 604 are positioned in a configuration corresponding to a first predetermined state such that the distance D₁ between the first optical element 602 and the center of the frame 601 and the distance D₂ between the second optical element 604 and the center of the frame 601 are substantially same to a distance D. In this configuration, as also shown in FIG. 6, the surface 621 of the first optical element 602 and the surface 641 of the second optical element 604 partially overlap to form a combined, or an effective, lens 600A with an effective focal power that is different from either of the focal power of the first optical element 602 and the focal power of the second optical element 604. The combined lens 600 can be characterized by a thickness T_{A} and a length L_{A}, in addition to the effective focal power. The first predetermined state thus is corresponding to a state where both, or at least one, of the first optical element 602 and the second optical element 604 are positioned eccentrically from the center of the frame 601, O. The intraocular lens system 600A at this state is in a relaxed state.

The frame 601 is elastic and adapted to be in contact with the lens capsule of the eye. The frame 601 can be chosen in a shape to fit to lens capsule equator so that the frame 601 and the lens capsule of the eye are closely in contact to each other. When the diameter of lens capsule of the eye changes due to age, for example, the lens capsule presses the frame 601 in a direction toward to the center of frame, O. The motion of the frame 601 causes the first optical element 602 and the second optical element 604 to move toward the center of the frame 601, O, from the first predetermined state to a second predetermined state, or a pressed state. At the pressed state, as shown in FIG. 6, the first optical element 602 and the second optical element 604 are substantially overlapping to each other so as to be substantially concentric with the center of the frame 601 to form a combined lens 600B with an effective focal power that is different from the effective focal power of the combined lens 600A at the first predetermined state. The combined lens 600B can be characterized by a thickness TB and a length LB, in addition to the effective focal power.

As shown in FIG. 6, the thickness T_{B} of the combined lens 600B at the pressed state is greater than the thickness T_{A} of the combined lens 600A at the relaxed state, and the length L_{B} of the combined lens 600B at the pressed state is smaller than the thickness L_{A} of the combined lens 600A at the relaxed state. Thus, the effective focal power of the combined lens 600B at the second predetermined state is larger than the effective focal power of the combined lens 600A at the first predetermined state, which allows the intraocular lens system 600 to be able to offer accommodation.

In one embodiment, the first optical component 602 comprises a lens that has a thickness T₁. The surface 621 of the lens 602 varies as a mathematical function of the distance from the center of the lens. The mathematical function can be a Gaussian distribution, a distribution that can be represented by a spherical-harmonic approximation, a distribution that is symmetrically decreasing with the distance from the center of lens as curves 712, 714 shown in FIG. 7 or the like. Likewise, the second optical component 604 comprises a lens that has a thickness T₂. The surface 641 of the lens 604 varies as a mathematical function of the distance from the center of the lens. The mathematical function can be a Gaussian distribution, a distribution that can be represented by a spherical-harmonic approximation, a distribution that is symmetrically decreasing with the distance from the center of lens as curves 712, 714 shown in FIG. 7 or the like. As an example, lenses with surfaces varying as a Gaussian distribution will be discussed in more detail below.

The frame 601 can have various configurations. For examples, the frame 601 can be a closed-loop structure. The closed-loop structure can be symmetrical to the center of the frame 601, O. In one embodiment, the frame 601 can be an annular or ring structure. In another embodiment, the frame 601 can be a multi-round-cornered structure. Alternatively, the frame can be an open-loop structure. Several configurations available to the frame 601 will be discussed in more detail below in connection with embodiments of the present invention as shown in FIG. 5 and FIG. 10.

Referring now to FIG. 12, an intraocular lens system 1200 has a first optical element 1210A and a second optical element 1210B positioned in a relaxed state. The intraocular lens system 1200 further has a frame 1212 to which the first optical element 1210A and the second optical element 1210B are attached. The frame 1212 is configured to fit into the lens capsule equator 102′. Additionally, the intraocular lens system 1200 has an optional supporting portion 1230 attached to the frame 1212. The supporting portion 1236 has a first end 1232, a second end 1234, and a surface 1236 defined between the first end 1232 and the second end 1234. The surface 1236 of the supporting portion 1230 has a curvature corresponding to the curvature of the lens capsule equator 102′ so as to allow the supporting portion to be positioned between the lens capsule and the frame 1212. The supporting portion 1230 thus can be utilized to hold the lens capsule open and provide a better fit between the intraocular lens system 1200 and the lens capsule. Additional supporting portions can be introduced for each of additional optical elements. For the embodiment shown in FIG. 12, for example, an additional optional supporting portion 1240 attached to the frame 1212 is introduced. The supporting portion 1240 has a first end 1242, a second end 1244, and a surface 1246 defined between the first end 1242 and the second end 1244. The surface 1246 of the supporting portion 1240 has a curvature corresponding to the curvature of the lens capsule equator 102′ so as to allow the supporting portion to be positioned between the lens capsule and the frame 1212.

Referring now to FIG. 5, an intraocular lens system 500 for implantation in an eye having a lens capsule and lens substance contained in the lens capsule is shown. The intraocular lens system 500 has a frame 512 having a center, O, and a plurality of lenses 510. The total number of the plurality of lenses 510 is an integer N. N can be any number equal to or grater than 2. For the embodiment shown in FIG. 5, N is chosen as six (6). Thus, the intraocular lens system 500 has lenses 510A, 510B, 510C, 510D, 510E, and 510F. Each of the plurality of lenses has a center, a surface and an edge, wherein the surface of each lens varies as mathematical function such as a Gaussian function of the distance from the center of the lens, being coupled to the frame 512 at the edge such that the center of each lens is at a distance from the center O of the frame 512. For instance, lens 510A has a center O₁, a surface 516A and an edge 514A. Lens 510A is coupled to the frame 512 at the edge 514A at a position 511A such that the center of lens 510A, O₁, is at a distance from the center of the frame 512, O. The plurality of lenses 510 at a first predetermined state, or relaxed state, are positioned such that the distances between the center of each lens and the center of the frame 512 are substantially same, and the surfaces of the lenses 510 partially overlap to form a combined lens 500 with an effective focal power. FIG. 8 schematically shows six (6) lenses 802 with a center O₁, 804 with a center O₂, 806 with a center O₃, 808 with a center O₄, 810 with a center O₅, and 812 with a center O₆ that are eccentrically and symmetrically positioned about the center O with an eccentric distance D.

The frame 512 is elastic and adapted to be in contact with and responsive to the lens capsule of the eye. The frame 512 can have various configurations. For the embodiment shown in FIG. 5, the frame 512 is a closed-loop structure that has a multi-round-corners 513A, 513B, 513C, 513D, 513E and 513F. One advantage of the multi-round-corners structure is that it allows less contact between the frame 512 and the lens capsule of the eye, which may be more suitable to people having sensitive eyes, for instance.

When the lens capsule of the eye presses the frame 512 in a direction toward to the center O of the frame 512, the motion of the frame 512 causes the plurality of the lenses to move toward the center of the frame 512 from the first determined state to a second predetermined state, or pressed state, in which the plurality of lenses 510A, 510B, 510C, 510D, 510E, and 510F are substantially overlapping to each other so as to be substantially concentric with the center of the frame 512 to form a combined lens with an effective focal power that is different from the effective focal power of the combined lens 500 at the first predetermined state. In general, the effective focal power of the combined lens 500 at the second predetermined state is larger than the effective focal power of the combined lens 500 at the first predetermined state that provides accommodation. If more lenses are introduced, the range of accommodation the intraocular lens system can offer is increased.

Referring now to FIG. 10, an intraocular lens system 1000 for implantation in an eye having a lens capsule and lens substance contained in the lens capsule is schematically shown. The intraocular lens system 1000 has a frame 1012 having a center, O, and a plurality of lenses 1010. The total number of the plurality of lenses 510 is an integer N. N can be any number equal to or grater than 2. For the embodiment shown in FIG. 10, N is chosen as six (6). Thus, the intraocular lens system 1000 has lenses 1010A, 1010B, 1010C, 1010D, 1010E, and 1010F. Each of the plurality of lenses has a center, a surface and an edge, wherein the surface of each lens varies as a mathematical function of the distance from the center of the lens, being coupled to the frame 1012 at the edge such that the center of each lens is at a distance from the center O of the frame 1012.

In comparison with the intraocular lens system 500 as shown in FIG. 5, one difference the intraocular lens system 1000 has is that the intraocular lens system 100 has a frame 1012 that is annular. One advantage of the annular structure is that it allows more contact between the frame 1012 and the lens capsule of the eye. Moreover, it is stable and easy to make. Additionally, for intraocular lens system 1000, each lens is coupled to the frame 1012 through an elastic thin wire, which allows some mobility capacity for the corresponding lens. For examples, lens 1010A is coupled to the frame 1012 through an elastic thin wire 1014A. There is no requirement to practice the present invention for a particular coupling method. A lens can be coupled to the frame through wire, by gluing, by molding, or the like methods know to people skilled in the art.

As discussed above and more below, the mathematical function can be a Gaussian distribution, a distribution that can be represented by a spherical-harmonic approximation, a distribution that is symmetrically decreasing with the distance from the center of lens, or the like.

If the mathematical function is chosen to be a Gaussian distribution, we call the corresponding lens as a Gaussian lens. The surface of a Gaussian lens, as shown in FIG. 7, in a three dimensional coordinate can be described as:

Z=te^{-a(x}^{²}^{+y}^{²}⁾

When six Gaussian lens overlap symmetrically, with an eccentric distance of d, which would be D as shown in FIG. 6, as shown in FIG. 8, their functions could be respectively expressed as:

Z₁=te^{-a[(x-d)}^{²}^{+y}^{²}^{]}

Z₂=te^{-a[(x+d)}^{²}^{+y}^{²}^{]}

Z₃=te^{-a[(x-d sin 30)}^{²}^{+(y-d cos 30)}^{²}^{]}

Z₄=te^{-a[(x+d sin 30)}^{²}^{+(y-d cos 30)}^{²}^{]}

Z₅=te^{-a[(x-d sin 30)}^{²}^{+(y+d cos 30)}^{²}^{]}

Z₆=te^{-a[(x+d sin 30)}^{²}^{+(y+d cos 30)}^{²}^{]}

The overlapped function in Z-X section would be:

$$Z = t {ⅇ}^{- {a ( x - d )}^{2}} + t {ⅇ}^{- {a ( x + d )}^{2}} + 2 t {ⅇ}^{- \frac{3}{4} {ad}^{2}} t {ⅇ}^{- {a ( x - \frac{1}{2} d )}^{2}} + 2 t {ⅇ}^{- \frac{3}{4} {ad}^{2}} t {ⅇ}^{- {a ( x + \frac{1}{2} d )}^{2}}$$

To calculate the radius of the overlapped function, one has to calculate its first and second derivative.

Z′=4at[d sinh(2adx)+d sinh(adx)−x cosh(2adx)−2x cosh(adx)]e^{-a(x}^{²}^{+d}^{²}⁾

$${Z}^{″} = 4 at \{ [ 2 {a ( x + d )}^{2} - 1 ] cosh ( 2 adx ) + [ 4 {a ( x + \frac{1}{2} d )}^{2} - 2 ] cosh ( adx ) - 4 adx {ⅇ}^{2 adx} - 4 adx {ⅇ}^{adx} \} {ⅇ}^{- a ( {x}^{2} + {d}^{2} )}$$

The focal power in diopter would be:

$$Diopter = ( n - 1 ) \vert \frac{{Z}^{″}}{{( 1 + {Z}^{′2} )}^{3 / 2}} \vert$$

At the central point,

Zₓ₌₀′=0

Z_{x=0,d=0}′=0

Zₓ₌₀″=12at(ad²−1)e^{-ad}^{²}

Z_{z=0,d=0}″=−12at.

As an example, if one chooses PMMA as optical material with refractive index of n=1.49, refractive index of aqueous is 1.336, then Gaussian function parameters will be given as follows:

t=2.1×10⁻⁵ m, and a=7.5×10⁵,

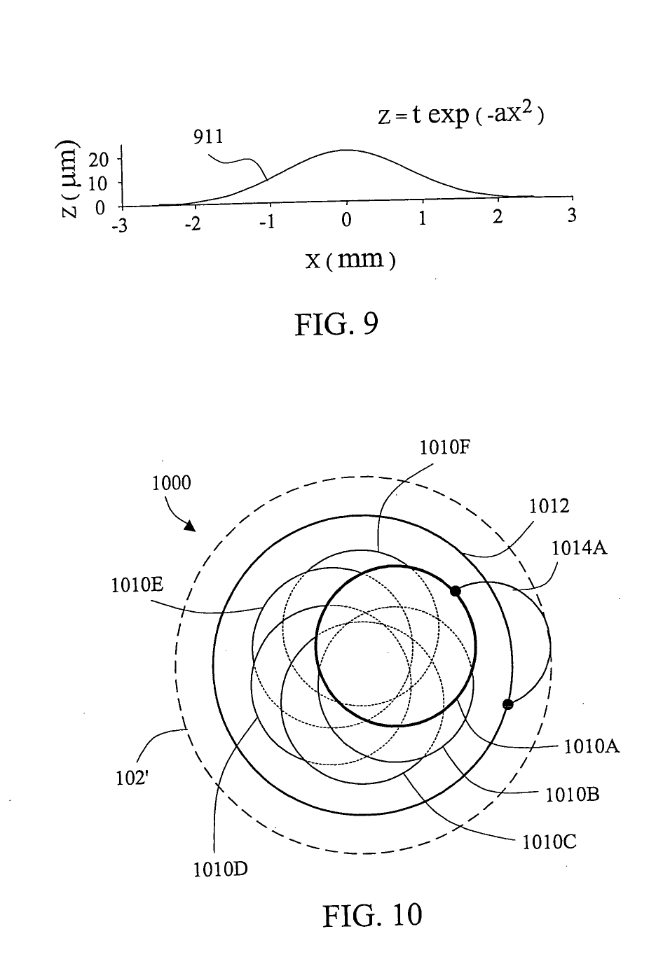

which means that the peak height of the Gaussian function is 21 μm, and the Gaussian function above 5% of the peak height spread about 4 mm in diameter as curve 911 shown in FIG. 9. If one adds 80 μm base thickness to this Gaussian function, then the thickness of each Gaussian lens would be only 100 μm.

The focal power of the combined intraocular lens system, i.e., when the lenses at the pressed state, is given by:

Diopter_{x=0,d=0}=30 D

Diopter_{x=0,d=0.5 mm}=20 D,

which means that when all six Gaussian lenses are overlapped concentrically in the embodiment as shown in FIG. 5, the combined lens 500 would have a focal power of 30D in the center. When each of the Gaussian lenses 510 is 0.5 mm off center, the combined lens 500 would have a focal power of 20D in the center. Thus, the present invention provides an intraocular lens system that can offer accommodation to a range that no one else has been able to offer.

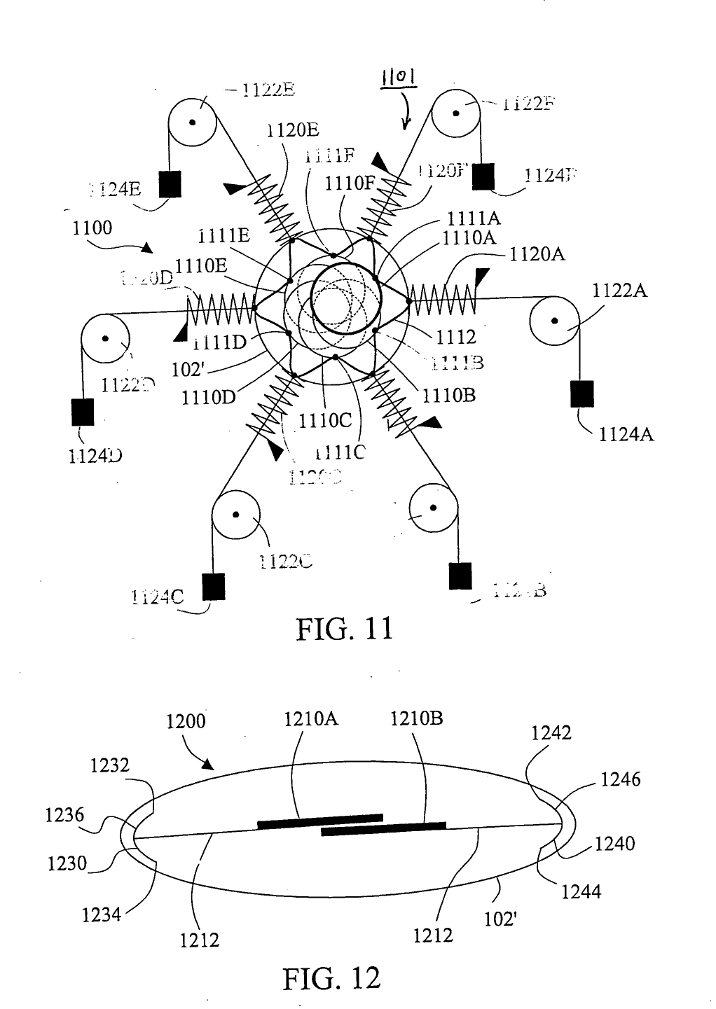

Additionally, a measuring system 1101 can be utilized to measure the force to move the lenses 1110A, 1110B, 1110C, 1110D, 1110E and 1111E in an IOL 1100 having frame 1112 the required distance(s) for a specific accommodation. As shown in FIG. 11, the measuring system 1101 has a measuring device for each lens. For example, the measuring system 1100 has spring 1120A, wheel 1122A, and weight 1124A for optical lens 1110A.

Optical ray tracing program can be utilized to precisely design the intraocular lens system according to the present invention. The image quality at different overlap conditions, and with different parameter selections can be analyzed. Through this type of analysis, the dioptic change of the intraocular lens system versus overlapping of the Gaussian lenses, as well as the best parameters for customizing the Gaussian lenses, can also be determined.

Other rotation curvatures such as sine, cosine, exponential, harmonics, or the like may also be calculated and/or utilized to compare with the Gaussian curve, so that one can determine which curve is likely to maximum accommodation.

Materials like PMMA can be used as the lens material. A computer-controlled diamond-turning machine can be used to fabricate the mold for molding the lenses. After the mold is made, large numbers of lenses can be manufactured by a heat-compressing procedure. This can be done through commercialized industrial services, such as Argus International, Ltd., at Scotts Valley, Calif.

Polypropylene, or polyamide can be utilized as the elastic frame material. Medical grade epoxy can be used to glue the lenses to the elastic frame. Heat compressing can also be utilized to couple the lenses to the frame. Moreover, different or alternate frame configurations can be designed and utilized to couple the lenses to the frame. For example, as discussed above, an alternative configuration utilizing a thin wire is shown at FIG. 10 according to another embodiment of the invention.

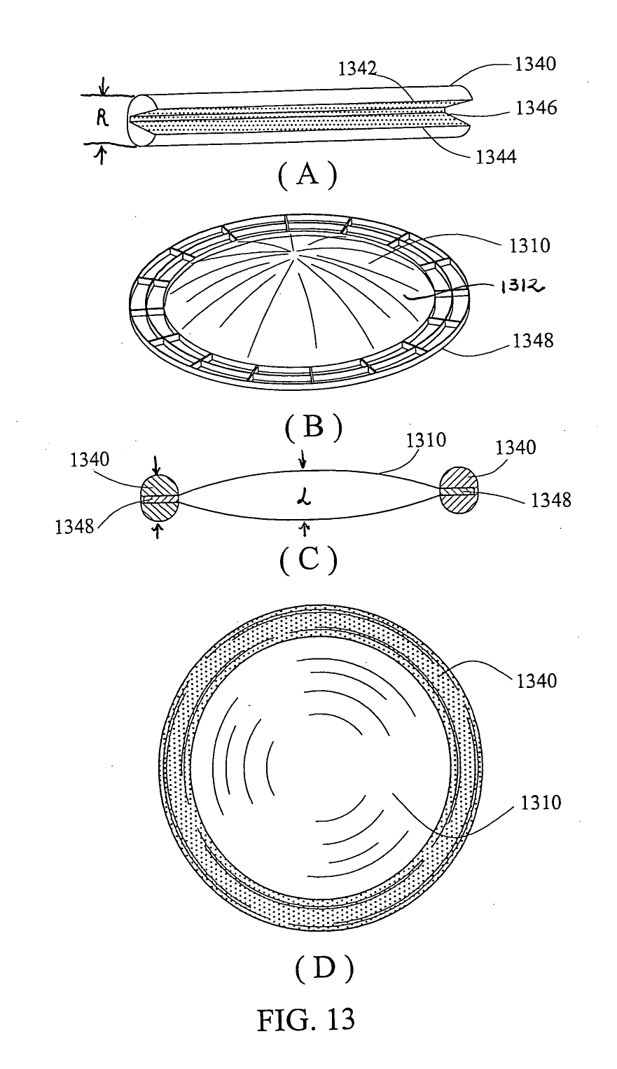

A potential problem for the intraocular lens systems according to the present invention is that when neighboring optical elements or lenses are overlapping to each other to form a combined lens, surfaces of the lenses that are optical sensitive may be negatively affected due to rubbing, scratching and/or pressing among them due to direct contact. Various options can be chosen, individually or in combination, to solve the problem. For example, proper lubrication can be applied to the surface of each lens. Moreover, optional structure may be introduced as well. As shown in FIGS. 13(A-D), a lens 1310 can be utilized to practice the present invention as a choice for optical elements. The lens 1310 has a thickness L and an optical surface 1312. The lens 1310 also has a peripheral frame 1348. A ridged edge portion 1340 has a first end 1342 and a second end 1344 defining a channel 1346 therebetween. The dimension of the channel 1346 is corresponding to that of the peripheral frame 1348 so that the peripheral frame 1348 can be received therein as best shown in FIG. 13C. As formed, the ridged edge portion 1340 is characterized by a dimension R that is no smaller than the thickness L of the lens 1310. Thus, when two lenses as shown in FIG. 13 overlap to each other, corresponding ridged edge portions will contact each other to prevent the optical sensitive surfaces of the lenses to rub against each other. The ridged edge portion 1340 can be made from different materials and have different configurations. For example, the ridged edge portion 1340 can comprise a strip made from elastic material compatible to the lens. Note that the strip should be narrow, and positioned only on the periphery of the lens so that they will not have significant influence on the refractive power of the intraocular lens system.

While there has been shown various embodiments of the present invention, it is to be understood that certain changes can be made in the form and arrangement of the elements of the system and steps of the intraocular lens system as would be known to one skilled in the art without departing from the underlying scope of the invention as is particularly set forth in the claims. Furthermore, the embodiments described above are only intended to illustrate the principles of the present invention and are not intended to limit the claims to the disclosed elements

What is claimed is:

1. An intraocular lens system for implantation in an eye having a lens capsule and lens substance contained in the lens capsule, comprising:
a. a frame 601 having a center;
b. a first optical element 602 with a focal power, the first optical element 602 having a center, a surface and an edge, wherein the first optical element 602 is coupled to the frame 601 at its edge such that the center of the first optical element 602 is at a distance from the center of the frame 601; and
c. a second optical element 604 with a focal power, the second optical element 604 having a center, a surface and an edge, wherein the second optical element 604 is coupled to the frame 601 at its edge such that the center of the second optical element 604 is at a distance from the center of the frame 601,
wherein the first optical element 602 and the second optical element 604 at a predetermined state are positioned such that the distance between the first optical element 602 and the center of the frame 601 and the distance between the second optical element 604 and the center of the frame 601 are substantially same, and the surfaces of the first optical element 602 and the second optical element 604 partially overlap to form a combined lens 600A with an effective focal power that is different from either of the focal power of the first optical element 602 and the focal power of the second optical element 604.

2. The intraocular lens system of claim 1, wherein the frame 601 is elastic and adapted to be in contact with the lens capsule of the eye.

3. The intraocular lens system of claim 2, wherein when the lens capsule of the eye presses the frame 601 in a direction toward to the center of frame, the motion of the frame 601 causes the first optical element 602 and the second optical element 604 to move toward the center of the frame 601 from the first predetermined state to a second predetermined state in which the first optical element 602 and the second optical element 604 are substantially overlapping to each other so as to be substantially concentric with the center of the frame 601 to form a combined lens 600B with an effective focal power that is different from the effective focal power of the combined lens 600A at the first predetermined state.

4. The intraocular lens system of claim 3, wherein the effective focal power of the combined lens 600B at the second predetermined state is larger than the effective focal power of the combined lens 600A at the first predetermined state.

5. The intraocular lens system of claim 2, wherein the frame 601 comprises a closed-loop structure.

6. The intraocular lens system of claim 5, wherein the closed-loop structure of the frame 601 is symmetrical to the center of the frame 601.

7. The intraocular lens system of claim 5, wherein the frame 601 comprises an annular structure.

8. The intraocular lens system of claim 5, wherein the frame 601 comprises a multi-round-cornered structure.

9. The intraocular lens system of claim 2, further comprising a supporting portion attached to the frame, the supporting portion having a first end, a second end, a surface defined between the first end and the second end, wherein the surface of the supporting portion has a curvature corresponding to the curvature of the lens capsule so as to allow the supporting portion to be positioned between the lens capsule and the frame.

10. The intraocular lens system of claim 1, wherein the first optical component comprises a lens that has a thickness.

11. The intraocular lens system of claim 10, wherein the surface of the lens varies as a mathematical function of the distance from the center of the lens.

12. The intraocular lens system of claim 11, wherein the mathematical function is a Gaussian distribution.

13. The intraocular lens system of claim 11, wherein the mathematical function is a distribution that can be represented by a spherical-harmonic approximation.

14. The intraocular lens system of claim 13, wherein the lens further comprises a ridged edge portion, the dimension of the ridged portion being comparable to the thickness of the lens.

15. The intraocular lens system of claim 1, wherein the second optical component comprises a lens that has a thickness.

16. The intraocular lens system of claim 15, wherein the surface of the lens varies as a mathematical function of the distance from the center of the lens.

17. The intraocular lens system of claim 16, wherein the mathematical function is a Gaussian distribution.

18. The intraocular lens system of claim 16, wherein the mathematical function is a distribution that can be represented by a spherical-harmonic approximation.

19. The intraocular lens system of claim 15, wherein the lens further comprises a ridged edge portion, the dimension of the ridged portion being comparable to the thickness of the lens.

20. An intraocular lens system for implantation in an eye having a lens capsule and lens substance contained in the lens capsule, comprising:
a. a frame 512 having a center; and
b. a plurality of lenses 510, total number being N, each of the plurality of lenses having a center, a surface and an edge, wherein the surface of each lens varies as a Gaussian function of the distance from the center of the lens, being coupled to the frame 512 at the edge such that the center of each lens is at a distance from the center of the frame 512,
wherein the plurality of lenses 510 at a first predetermined state are positioned such that the distances between the center of each lens and the center of the frame 512 are substantially same, and the surfaces of the lenses partially overlap to form a combined lens 500 with an effective focal power.

21. The intraocular lens system of claim 20, wherein the frame 512 is elastic and adapted to be in contact with and responsive to the lens capsule of the eye. The intraocular lens system of claim 21, wherein when the lens capsule of the eye presses the frame 512 in a direction toward to the center of the frame 512, the motion of the frame 512 causes the plurality of the lenses to move toward the center of the frame 512 from the first determined state to a second predetermined state in which the plurality of lenses are substantially overlapping to each other so as to be substantially concentric with the center of the frame 512 to form a combined lens with an effective focal power that is different from the effective focal power of the combined lens 500 at the first predetermined state.

22. The intraocular lens system of claim 22, wherein the effective focal power of the combined lens at the second predetermined state is larger than the effective focal power of the combined lens at the first predetermined state.

23. The intraocular lens system of claim 20, wherein the plurality of lenses 510 are positioned such that the centers of the plurality of lenses 510 are symmetrical about the center of the frame 512.

24. The intraocular lens system of claim 20, wherein N is an integer that is not smaller than 2.

25. An intraocular lens system for implantation in an eye having a lens capsule and lens substance contained in the lens capsule, comprising:
a. a frame 512 having a center; and
b. a plurality of lenses 510, total number being N, each of the plurality of lenses having a center, a surface and an edge, wherein the surface of each lens varies as a mathematical function of the distance from the center of the lens, being coupled to the frame 512 at the edge such that the center of each lens is at a distance from the center of the frame 512;
wherein the plurality of lenses 510 at a first predetermined state are positioned such that the distances between the center of each lens and the center of the frame 512 are substantially same and the surfaces of the lenses partially overlap to form a combined lens 500 with an effective focal power.

26. The intraocular lens system of claim 26, wherein the frame 512 is elastic and adapted to be in contact with and responsive to the lens capsule of the eye.

27. The intraocular lens system of claim 27, wherein when the lens capsule of the eye presses the frame 512 in a direction toward to the center of the frame 512, the motion of the frame 512 causes the plurality of the lenses to move toward the center of the frame 512 from the first predetermined state to a second predetermined state in which the plurality of lenses are substantially overlapping to each other so as to be concentric with the center of the frame 512 to form a combined lens with an effective focal power that is different from the effective focal power of the combined lens 500 at the first predetermined state.

28. The intraocular lens system of claim 28, wherein the effective focal power of the combined lens at the second predetermined state is larger than the effective focal power of the combined lens at the first predetermined state.

29. The intraocular lens system of claim 26, wherein the plurality of lenses 510 are positioned such that the centers of the plurality of lenses 510 are symmetrical about the center of the frame 512.

30. The intraocular lens system of claim 26, wherein N is an integer that is no smaller than 2.

31. The intraocular lens system of claim 26, wherein the mathematical function is a Gaussian distribution.

32. The intraocular lens system of claim 26, wherein the mathematical function is a distribution that can be represented by a spherical-harmonic approximation.

33. The intraocular lens system of claim 26, wherein the mathematical function is a distribution that is symmetrically decreasing with the distance from the center of lens.

34. The intraocular lens system of claim 26, wherein each of the plurality of lenses has a thickness and further comprises a ridged edge portion, the dimension of the ridged portion being at least comparable to the thickness of that lens.

35. The intraocular lens system of claim 26, further comprising at least one supporting portion attached to the frame, the supporting portion having a first end, a second end, a surface defined between the first end and the second end, wherein the surface of the supporting portion has a curvature corresponding to the curvature of the lens capsule so as to allow the supporting portion to be positioned between the lens capsule and the frame.
